(19) United States
(12) Patent Application Publication
(10) Pub. No.: US 2011/0244052 A1
(43) Pub. Date: Oct. 6, 2011

(54) POLYUNSATURATED FATTY ACIDS FOR IMPROVING VISION

(75) Inventor: Asgeir Saebo, Eidsnes (NO)

(73) Assignee: PHARMA MARINE AS, Alesund (NO)

(21) Appl. No.: 13/055,289

(22) PCT Filed: Jul. 24, 2009
(86) PCT No.: PCT/GB2009/001848

§ 371 Date: Jun. 14, 2011

(30) Foreign Application Priority Data
Jul. 24, 2008 (GB) 0813599.8

(51) Int. Cl. A61K 35/60; A61K 31/201; A61K 31/202; A61K 35/56; A61P 27/10; A61K 35/12

(52) U.S. Cl. 424/523; 514/560; 424/522

(57) ABSTRACT

The invention provides an oral nutraceutical or pharmaceutical composition comprising at least one polyunsaturated fatty acid (PUFA) or a derivative thereof for use in treating defects of vision, e.g. long-sightedness (hyperopia). In some embodiments, the PUFA in the composition is provided by an oil purified from squid.

This invention relates to a method of improving vision in a subject, in particular to a method of reducing or reversing the progression of long-sightedness, comprising administering polyunsaturated fatty acid (PUFA) derivatives, especially derivatives of omega 3 fatty acids such as eicosapentaenoic acid (EPA) and docosahexaenoic acid (DHA), to the subject and also to compositions for use in said method.

Poor, limited or ailing vision is a problem that affects almost everybody at some stage during their lifetime. Failing vision is a particular problem amongst the elderly and addressing this problem will become progressively more important in our increasingly aging societies. Examples of conditions that affect vision include macular degeneration, particularly prevalent amongst those over the age of 50 (age-related macular degeneration—AMD) and long-sightedness, which can be caused by imperfections in the eye (hyperopia) or by reduction in the amplitude of accommodation of the eye, often associated with changes to the lens and reduced muscle function. Accommodation is the ability of the lens of the eye to change shape, allowing for a change in focus from far to near. The lens, which is flexible, becomes more round to bring near objects into focus and flattens to bring distant objects into focus. With age, the lens of the eye loses some of its flexibility, reducing the ability to accommodate. This usually happens around age 45 and is known as presbyopia.

In particular, long-sightedness tends to develop over time in eyes that are otherwise healthy. From the age of approximately 40 years people become increasingly dependent on corrective lenses or surgical treatments to maintain an acceptable focus on near objects. The strength of corrective lenses required to offset the increasing long-sightedness that occurs with advancing age will be increased by a factor of at least about +1 dioptre every 10 years.

Furthermore, people who experience difficulty focussing on near objects often find that the condition varies in severity from day to day. This results in difficulties in performing daily tasks, even if corrective lenses (usually of a fixed prescription) are used.

Long-chain polyunsaturated fatty acids represent an important food supplement. Mammals lack the ability to introduce double bonds in fatty acids beyond carbons 9 and 10 and hence certain fatty acids (e.g. linoleic acid) are essential supplements for humans. In the body, essential fatty acids are primarily used to produce substances that regulate a wide range of functions, including blood pressure, blood clotting, blood lipid levels, the immune response, and the inflammation response to injury or infection.

References herein to fatty acids are intended to cover fatty acid derivatives, such as salts and esters as well as glycerides (e.g. triglycerides) and phospholipids thereof.

An important sub-class of polyunsaturated fatty acids are the omega 3 fatty acids, which all have a carbon-carbon double bond in the omega 3 position, i.e. the third carbon-carbon bond from the terminal methyl end (ω) of the carbon chain. The nutritionally important omega 3 fatty acids include α-linolenic acid (ALA), eicosapentaenoic acid (EPA) and docosahexaenoic acid (DHA).

Omega 3 fatty acids are available from a number of natural sources. These include higher animals, especially fish such as cod and salmon and mammals such as seals which are rich in DHA and EPA. Squid is a particularly rich source of DHA and EPA as well as other polyunsaturated fatty acids. DHA is also found in algae such as Crypthecodinium cohnii. Botanical sources of the omega 3 fatty acids include kiwifruit and flax (linseed) as well as nuts such as walnuts and pecans. The botanical sources tend to be high in ALA. The extraction and purification of fatty acids from biological sources is well described in the scientific literature.

Recommendations for the daily intake of omega 3 fatty acids in humans vary, although the US National Institutes of Health recently recommend a total daily intake of 650 mg of EPA and DHA, and 2.22 g of ALA. The US Federal Drug Agency recommends no more than 2 g per day of omega 3 fatty acids from nutritional supplements. However, several studies have suggested that a daily intake of omega 3 fatty acids of between 2 g and 10 g per day is not detrimental to health. It is believed that ALA may be converted to EPA and then to DHA in humans and women are reported to have a higher ALA conversion efficiency than men. It may thus be necessary to tailor the dosage of omega 3 fatty acids to the sex of the patient as well as to other factors, such as their age and medical history.

Dietary supplementation of omega 3 fatty acids has been linked with a reduced risk of coronary heart disease, ischemic and thrombotic stroke as well as some cancers. Certain mental disorders, such as aggression and schizophrenia, may be ameliorated by omega 3 supplements. Most studies into the above conditions have used fish oil supplements as the source of omega 3 fatty acids.

Omega 3 fatty acids are also known to play a role in vision. The retina is rich in long-chain fatty acids, the phospholipid component of which may contain a significant amount of DHA. Dietary supplementation with long-chain polyunsaturated fatty acids (e.g. DHA) has been associated with a decreased likelihood of age-related macular degeneration and has also been linked to an improvement in dry eye syndrome in women.

By “palliative” as used herein is meant a treatment regime for a condition that relieves the symptoms of the condition without necessarily effecting a cure. In the case of defects of vision associated with loss of ocular muscle strength, it is usually considered inevitable that the defects will become progressively worse with age. In the case of long-sightedness, e.g. caused by hyperopia, a successful palliative treatment is one which achieves a +0.5, especially a +1, dioptre reduction in the strength of prescription for corrective lenses in the subject undergoing treatment. The treatment may, in extremis, return the vision to a state wherein corrective lenses are no longer required. A successful palliative treatment is also defined as one which perceptibly reduces the variability of vision (e.g. throughout the day or in differing light levels). The palliative treatment according to the invention may be continued indefinitely; however, an effective dosage is one which results in a successful treatment over a defined period, for example over 2 weeks, over 6 weeks or over a period of less than 24 weeks.

The inventor of the present invention has found that supplementing the diet with polyunsaturated fatty acids, particularly omega 3 fatty acids, can improve the vision of the subject taking the supplement. It has been surprisingly found that certain conditions which have not previously been linked with fatty acid intake, such as long- and short-sightedness, may be improved by supplementation of the diet as described. Without wishing to be bound by theory, it is postulated that dietary fatty acid supplementation may improve the muscular function of the intra- and extra-ocular muscles, thereby giving rise to this unexpected and until now unknown effect of fatty acid administration. Dietary fatty acid supplementation may also improve the flexibility and/or clarity of the lens of the eye. In particular, the inventor has found that the vision of a long-sighted individual taking the PUFA-containing compositions of the invention may be improved by a factor of +1 to +2 dioptres, thus leading to a potential postponement of the age at which corrective lenses may be required of between about 10 and 20 years.

Accordingly, in one aspect the present invention provides a method of palliative treatment of defective vision of a human or non-human mammalian subject in need thereof comprising orally administering an effective dose of at least one polyunsaturated fatty acid (PUFA) to said subject wherein the defect of vision to be improved is other than age-related macular degeneration and dry eye syndrome. The palliative treatment of the invention serves to delay or prevent the eyesight of the subject from worsening and also to prevent the variation in visual function experienced by subjects over time, e.g. daily. In some cases, the palliative treatment of the invention may not just delay or prevent the worsening of the eyesight of the subject but may in fact improve the eyesight of the subject. One example of an indication of improvement in eyesight is a reduction in the strength of a prescription required for corrective lenses.

Particularly contemplated is a method of prevention or treatment of a defect of vision in a human or non-human mammalian subject which defect may be improved by potentiating the intra- or extra-ocular musculature of said subject.

Also contemplated is a method of prophylactic treatment of a human or non-human mammalian subject to delay or prevent the onset of vision defects which method comprises orally administering to said subject an effective dose of at least one polyunsaturated fatty acid (PUFA) or derivative thereof.

The defect of vision to be treated is preferably hyperopia (long-sightedness), myopia (short-sightedness) or presbyopia, most preferably hyperopia. In one embodiment, the defect of vision to be treated is other than myopia.

In a preferred embodiment, the subject is human, more preferably a human male. The palliative, prophylactic or curative treatment of the invention is preferably effected on a human subject being at least 20 years old, preferably at least 30 years old, e.g. between 40 and 70 years old.

A further aspect of the invention concerns compositions (e.g. nutraceutical or pharmaceutical compositions) for administration in the treatment methods described herein.

In particular, the invention provides an oral nutraceutical or pharmaceutical composition comprising at least one polyunsaturated fatty acid (PUFA) or a derivative thereof for use in treating a defect of vision in a human or non-human mammalian subject which defect may be improved by potentiating the intra- or extra-ocular musculature of said subject.

Also provided, according to the invention, is an oral nutraceutical or pharmaceutical composition comprising at least one polyunsaturated fatty acid (PUFA) or a derivative thereof for use in a palliative treatment of defective vision of a human or non-human mammalian subject comprising orally administering said composition to said subject and wherein the defect of vision to be improved is other than age-related macular degeneration and dry eye syndrome.

Also provided, according to the invention, is an oral nutraceutical or pharmaceutical composition comprising at least one polyunsaturated fatty acid (PUFA) or a derivative thereof for use in a method of prophylactic treatment of a human or non-human mammalian subject to delay or prevent the onset of vision defects which method comprises orally administering said composition to said subject.

In preferred embodiments the at least one PUFA is an omega 3 PUFA which is preferably selected from alpha-linolenic acid (ALA), stearidonic acid, eicosatetraenoic acid, eicosapentaenoic acid (EPA), docosapentaenoic acid (DPA), docosahexaenoic acid (DHA) and nisinic acid, particularly preferably selected from EPA or DHA and is most preferably DHA. In another preferred embodiment of the invention, the at least one PUFA is EPA. The PUFA of the invention may typically be administered as a salt or an ester derivative of the acid. Particularly preferred for administration are the triglyceride and/or phospholipid derivatives of the acid.

Fatty acid compositions of PUFA derivatives comprising a plurality of fatty acid groups (for example triglycerides or phospholipid derivatives) may be made up of the same of different fatty acids, i.e. the fatty acid groups of each derivative may be the same or different. Preferably a triglyceride derivative will comprise three omega 3 fatty acid groups and a phospholipid derivative will comprise two omega 3 fatty acid groups.

Preferred sources of PUFAs for use according to the invention are anchovies, sardines, cod, salmon, seal and squid. Squid, e.g. squid visceral material, is a particularly preferred source of omega 3 fatty acids for use according to the invention. In one embodiment, the PUFA source is a non-exoskeletal animal source.

The compositions of the invention may comprise one or more oxidation inhibitors to delay the oxidation of the fatty acids. The composition may also be encapsulated, either micro-encapsulated or encapsulated in a larger scale, e.g. for direct administration in a capsule. Methods for encapsulating and micro-encapsulating fatty acids and fatty acid compositions are well known in the art.

The compositions for oral administration may typically be in the form of a liquid dosage (to be administered e.g. by the spoonful), a powder or tablet or a capsule. A bulk dosage form will typically consist of sufficient unit doses (e.g. capsules or tablets) to provide the required quantity of composition. The number and size of these unit doses will depend on the final dose required and also on the tolerances of the subject taking the unit doses. Preferably the unit dose for oral administration has a cylindrical or ellipsoidal shape. It is generally found that subjects will not tolerate unit doses of greater than about 1 g to 2 g and the young and elderly, with whom compliance is a greater problem, will generally require smaller still unit doses, e.g. 500 mg. It is, however, preferable to administer the required quantity of composition in as few unit doses as possible and a balance may need to be struck between the size of the unit dose and the number of units to be administered.

The quantity of PUFA for administration will depend on the palliative treatment required, for example on the nature of the defect of vision, but will generally involve a dosage of between 0.5 g and 10 g of the at least one PUFA. In a preferred embodiment of the invention, the at least one PUFA is administered in a dosage of greater than about 0.1 g, preferably greater than about 1 g and particularly preferably greater than about 2 g. Particularly preferred are dosages of the at least one PUFA of less than about 10 g, preferably less than about 6 g, e.g. between 0.1 g and 10 g and particularly preferably between 1 g and 4 g. These dosages are calculated on the basis of the PUFA as a free fatty acid. In one preferred embodiment the above-mentioned dosages represent the dose of active PUFA in the administered composition. Alternatively, if multiple fatty acid components (especially active components) are present in the composition for administration, each component may be present in the above-mentioned amounts.

The dosage regime for the compositions of the invention may comprise the administration of the daily dosage at one time (i.e. after a morning meal) or at a plurality of occasions throughout the day (i.e. half of the daily dosage in the morning and half in the evening). If a multiple time-point administration regime is to be followed, the daily dosage is preferably divided into dosages that provide the required amount of composition at each time-point. For example, a routine of three equal dosages during the day might be conveniently administered as three unit dosages each of one third of the daily dose, or as six unit dosages each of one sixth of the daily dose, etc. In a preferred embodiment, a composition according to the invention is adapted for administration in a single daily dosage.

Preferably at least 5% by weight of the composition comprising the at least one PUFA (excluding the weight of a coating on a capsule, or the like) is polyunsaturated fatty acid, especially at least 10% and most preferably at least 15%.

If the composition comprises a partially purified oil, e.g. a fish oil, a squid oil, a seal oil etc., then the proportion of each active fatty acid component in the oil should be calculated to determine the total dosage required. For example, a composition of 6 g of oil having 35% of active omega 3 fatty acids could be effectively replaced by a composition of 3 g of oil having 70% of the same active omega 3 fatty acids. From a practical consideration, therefore, the active components of the composition should ideally be as pure as possible and in as high a concentration as possible to minimise the amount of composition required for administration. Accordingly, the at least one PUFA composition may be a substantially pure PUFA composition, e.g. at least about 75% pure, at least about 85% pure, at least about 90% pure, at least about 95% pure or at least about 98% pure.

Substantially pure encapsulated omega 3 fatty acid compositions, particularly substantially pure EPA and DHA compositions, as well as the salt, ester, glyceride or phospholipid derivatives thereof form a further aspect of the invention. Preferably the omega 3 fatty acid is EPA or DHA, or a salt, ester, glyceride or phospholipid derivatives thereof, which is at least about 85%, preferably at least about 95% pure. In a preferred embodiment, the encapsulated substantially pure omega 3 fatty acid or a salt, ester, triglyceride or phospholipid derivative thereof is suitable for use as a medicament. An encapsulated composition comprising substantially pure EPA and/or DHA from squid is particularly preferred. The use of an encapsulated squid oil, preferably comprising at least one PUFA or derivative thereof as defined herein, as a medicament is itself novel and forms a further aspect of the invention. Mixtures of fatty acids for administration may be encapsulated together—i.e.

mixed together, or encapsulated non-mixed—wherein the dosage form would comprise one or more capsules of each substantially pure component separately.

In one embodiment of the invention, the composition for administration may comprise one or more other vision enhancing agents, e.g. vitamins such as vitamins A, D, E, mineral supplements such as iron, magnesium and zinc and other active compounds such as carotenoids, e.g. lutein and zeaxanthin.

A further aspect of the invention is directed towards nutraceutical and pharmaceutical preparations comprising the compositions described herein. According to this aspect, the invention provides a pharmaceutical preparation comprising a composition as hereinbefore defined and one or more excipients, carries, or diluents. The active compositions are typically combined, e.g. non-chemically, with known excipients such as binders, gelling agents, lubricants, flow agents, colours, antioxidants, flavours, stabilisers etc. to form the unit dosage. These unit dosages may then be coated or otherwise finished the increase their storage stability, e.g. their resistance to oxidation. Suitable excipients and coatings as well as methods for the preparation of pharmaceutical preparations are well known in the art.

A kit is also provided which comprises one or more of the compositions described herein, or the nutraceutical or pharmaceutical preparations thereof, preferably adapted to provide daily dosages of the composition in as many unit dosages as make up the daily dosage. The kit comprising instructions for the administration of said composition in a method of treatment of a defect of vision in a human or non-human mammalian subject as hereinbefore described forms a further aspect of the invention.

The invention will now be further described with reference to the following non-limiting Examples:

### EXAMPLE 1

### Effect of Omega 3 Fatty Acids on Long-sightedness

Two male human subjects took a nutraceutical supplement of 6 g per day of oil. The oil contained 35% (by weight of oil) omega 3 fatty acids. The oil contained 18% EPA (by weight of oil) and 12% DHA (by weight of oil), the content of fatty acids being calculated as weight of free fatty acid by weight of oil. The supplement also contained minor amounts of alpha-linolenic acid and stearidonic acid. Fatty acid composition of the oil was calculated by area Gas Chromatography analysis and indicated that 6 g of oil would provide approximately 1034 mg EPA and 689 mg DHA (as free fatty acids) after hydrolysis and adsorption

After three weeks both subjects observed an improvement in the ability of their eyes to focus on short distances. One of the subjects (aged 50) had regularly been using reading glasses (+1) for short-distance work, but experienced that reading was not difficult without the glasses after taking the supplement. The other subject (aged 45) was able to delay the onset of using reading glasses after taking the supplement. In each case the improvement was a factor of approximately +1 dioptre.

An improvement in vision of up to +2 dioptre is predicted in some cases.

### EXAMPLE 2

### Preparation of a Purified Oil from Squid

3000 grams of frozen liver from squid is freeze dried in a laboratory freeze drier. The freeze dried material is then subjected to a supercritical fluid extraction (SFE) process. Carbon dioxide is flushed through the material at a pressure of 500 bars. The quantity corresponds to 12 kg CO₂ per kilo of material. 450 grams of lipids are collected in the receiver after depressurization. Then 20% ethanol is added to the solvent, and another 160 grams (after removal of ethanol) of lipids is collected. The neutral fraction is distilled twice in a molecular distillation plant after a degassing step to obtain a vacuum of 0.001 mbar. The free fatty acids and most of the cholesterol is removed in the distillation at 190° C. The neutral fraction is then subjected to bleaching in a batch reactor using 1% of activated bleaching clay at 60° C. for 45 minutes. The remaining oil is filtered to remove the clay.

The ethanol in the polar fraction is removed in a rotary evaporator down to approximately 20% ethanol content. Then this fraction is combined with the distilled and bleached neutral fraction, e.g. as an equal amount by weight, to yield a final product high in omega 3 and high in phospholipids.

### EXAMPLE 3

### Nutraceutical Composition Comprising Squid Oil

A purified oil from squid visceral material which is suitable for human consumption is obtained, e.g. by a process according to Example 2. The oil comprises (percentages by weight of oil calculated on the basis of the fatty acid content as free fatty acids):

| | |
|---|---|
| Omega 3 fatty acids | 36% of which |
| EPA | 14% (by weight of oil) |
| DHA | 20% (by weight of oil) |

The squid oil is filled into capsules containing approximately 1 g of oil per capsule. The capsules are packaged with instructions for adults to take 3 capsules per day.

### EXAMPLE 4

### Nutraceutical Composition Comprising Squid Oil Concentrate

A purified fatty acid ethyl ester concentrate from squid visceral material which is suitable for human consumption is obtained, e.g. by a process according to Example 2. The concentrate comprises (percentages by weight of oil calculated on the basis of the fatty acid content as free fatty acids):

| | |
|---|---|
| Omega 3 fatty acids | 68% of which |
| EPA | 15% (by weight of oil) |
| DHA | 50% (by weight of oil) |

The squid oil is filled into capsules containing approximately 1 g of oil per capsule. The capsules are packaged with instructions for adults to take 2 capsules per day.

1. A method of treating long-sightedness (hyperopia) in a subject, the method comprising administering to said subject an effective amount of an oral nutraceutical or pharmaceutical composition comprising at least one polyunsaturated fatty acid (PUFA) or a derivative thereof.

2. The composition of claim 1, wherein said composition comprises oil purified from squid, wherein the at least one PUFA is provided by said oil.

3. The composition of claim 1, wherein said composition comprises oil purified from anchovies, sardines, salmon, cod or seal, wherein the at least one PUFA is provided by said oil.

4. A method of treating a defect of vision in a human or non-human mammalian subject which defect may be improved by potentiating the intra- or extra-ocular musculature of said subject, the method comprising administering to said subject an oral nutraceutical or pharmaceutical composition comprising at least one polyunsaturated fatty acid (PUFA) or a derivative, wherein the at least one PUFA is provided by an oil purified from squid.

5. (canceled)

6. A method of prophylactic treatment of a human or non-human mammalian subject to delay or prevent the onset of vision defects, which method comprises orally administering to said subject an oral nutraceutical or pharmaceutical composition comprising at least one polyunsaturated fatty acid (PUFA) or a derivative thereof, wherein the at least one PUFA is provided by an oil purified from squid.

7. The method of claim 4 for treating hyperopia (long-sightedness), myopia (short-sightedness) or presbyopia, preferably hyperopia, in a human subject.

8. The method of claim 1, wherein the at least one PUFA or derivative thereof is an omega 3 PUFA, preferably selected from alpha-linolenic acid (ALA), stearidonic acid, eicosatetraenoic acid, eicosapentaenoic acid (EPA), docosapentaenoic acid (DPA), docosahexaenoic acid (DHA) and nisinic acid, particularly preferably selected from EPA or DHA, and wherein said derivative thereof is selected from a salt, an ester, a glyceride or a phospholipid.

9. The method of claim 1, said composition further comprising at least one other vision-enhancing agent.

10. The method of claim 1, wherein said at least one PUFA is administered in a dosage of between 0.1 g and 10 g, calculated on the basis of the PUFA as a free fatty acid.

11. The method of claim 1, wherein said composition is administered in a single daily dosage.

12-14. (canceled)

15. A method of palliative treatment of defective vision of a human or non-human mammalian subject comprising orally administering an effective dose of at least one polyunsaturated fatty acid (PUFA) to said subject wherein the defect of vision to be improved is other than age-related macular degeneration and dry eye syndrome, wherein the at least one PUFA is provided by an oil purified from squid.

16. A method of treatment of hyperopia (long-sightedness), myopia (short-sightedness) or presbyopia comprising orally administering an effective dose of a polyunsaturated fatty acid (PUFA) or derivative thereof to a mammalian subject in need thereof.

17-18. (canceled)

19. The method of claim 4 wherein the at least one PUFA or derivative thereof is an omega 3 PUFA, preferably selected from alpha-linolenic acid (ALA), stearidonic acid, eicosatetraenoic acid, eicosapentaenoic acid (EPA), docosapentaenoic acid (DPA), docosahexaenoic acid (DHA) and nisinic acid, particularly preferably selected from EPA or DHA, and wherein said derivative thereof is selected from a salt, an ester, a glyceride or a phospholipid.

20. The method of claim 4, said composition further comprising at least one other vision-enhancing agent.

21. The method of claim 4, wherein said at least one PUFA is administered in a dosage of between 0.1 g and 10 g, calculated on the basis of the PUFA as a free fatty acid.

22. The method of claim 4, wherein said composition is administered in a single daily dosage.

23. The method of claim 6 for treating hyperopia (long-sightedness), myopia (short-sightedness) or presbyopia, preferably hyperopia, in a human subject.

24. The method of claim 6 wherein the at least one PUFA or derivative thereof is an omega 3 PUFA, preferably selected from alpha-linolenic acid (ALA), stearidonic acid, eicosatetraenoic acid, eicosapentaenoic acid (EPA), docosapentaenoic acid (DPA), docosahexaenoic acid (DHA) and nisinic acid, particularly preferably selected from EPA or DHA, and wherein said derivative thereof is selected from a salt, an ester, a glyceride or a phospholipid.

25. The method of claim 6, said composition further comprising at least one other vision-enhancing agent.

26. The method of claim 6, wherein said at least one PUFA is administered in a dosage of between 0.1 g and 10 g, calculated on the basis of the PUFA as a free fatty acid.

27. The method of claim 6, wherein said composition is administered in a single daily dosage.
